(19) United States
(12) Patent Application Publication
(10) Pub. No.: US 2021/0138400 A1
(43) Pub. Date: May 13, 2021

(54) COMPOUND FILTER MODULE FOR VEHICLE AIR CLEANER

(71) Applicant: WaytoMake Co., Ltd., Gyeongsangbuk-do (KR)

(72) Inventor: Suejin JEON, Gyeongsangbuk-do (KR)

(73) Assignee: WaytoMake Co., Ltd., Gyeongsangbuk-do (KR)

(21) Appl. No.: 16/914,041

(22) Filed: Jun. 26, 2020

(30) Foreign Application Priority Data
Nov. 13, 2019 (KR) 10-2019-0145275

(51) Int. Cl. B01D 53/75; B01D 53/00; B01D 53/88; B01D 53/04; B01D 53/06; B01D 46/12; B01D 46/00; B01D 53/86; B01J 21/06; B01J 21/08; B01J 35/00; B01J 37/00; B01J 37/02; B01J 20/18; A61L 9/20; B60H 3/06

(52) U.S. Cl. CPC B01D 53/75; B60H 2003/0675; B01D 53/885; B01D 53/0415; B01D 53/06; B01D 46/12; B01D 46/0023; B01D 46/0036; B01D 46/0028; B01D 46/0038; B01D 53/8671; B01J 21/063; B01J 21/08; B01J 35/0013; B01J 35/004; B01J 37/0018; B01J 37/0215; B01J 20/18; A61L 9/205; B60H 3/0608; B01D 2267/30; B01D 2279/40; B01D 2259/804; B01D 2255/802; B01D 2253/108; B01D 2255/20707; B01D 2257/504; B01D 2257/708; A61L 2209/14; A61L 2209/12; B01D 53/007

(57) ABSTRACT

A compound filter module includes an upper body in which at least one side of an upper portion is opened, an UV irradiation unit which is coupled to a lower portion of the upper body, includes a plurality of UV LEDs, and is configured to perform irradiation of UV from a lower body side, a disk-shaped photocatalyst cartridge which is located below the UV irradiation unit and includes a plurality of photocatalytic beads including a photocatalyst reacting with UV emitted from the UV irradiation unit, a disk-shaped zeolite cartridge which is located below the photocatalyst cartridge and includes a plurality of zeolite beads adsorbs harmful substances from the polluted air sucked through the lower body, and the lower body which is located below the zeolite cartridge and in which at least one side of a lower portion is formed to be opened, in which each of the photocatalyst cartridge and the zeolite cartridge includes at least one through hole which is formed in an up-down direction.

### CROSS-REFERENCE TO RELATED APPLICATION

This application claims the benefit of Korean Patent Application No. 10-2019-0145275, filed on Nov. 13, 2019, which is hereby incorporated by reference in its entirety.

### BACKGROUND

### Field of the Disclosure

The present disclosure relates to a compound filter module for a vehicle air cleaner, and more particularly, to a compound filter module for a vehicle air cleaner capable of increasing polluted air throughout to improve air purification efficiency while sucking, absorbing, and decomposing polluted air existing in a vehicle so as to discharge purified air.

### Description of the Background

Various pollutions exist inside a vehicle, including fine dust and polluted air from outside vehicle, carbon oxide generated by breathing of a driver and a passenger, hydrocarbon, carbon oxide, sulfur dioxide, and a nitrogen oxide generated by a vehicle interior material and a driving element, and studies have shown that air pollution inside the vehicle can increase up to 15 times when the vehicle is stopped due to a traffic jam or traffic jam.

In a case where air quality inside the vehicle deteriorates as much as damages suffered by a pedestrian due to air pollution generated by a diesel vehicle or the like, and the passengers including the driver are largely damaged by polluted air in a closed space.

The polluted air is a cause of various diseases ranging from asthma, chronic obstructive pulmonary disease, heart disease or stroke, and thus, it is very important social issue to remove air pollution inside the vehicle which is rapidly increasing with industrial development.

In order to solve the above-described problems, various vehicle air cleaners usable in a vehicle have been developed, and an example of the related art, there is a “filter assembly for an air cleaner” of Korean Patent No. 10-2039273.

The related art relates to the filter assembly for an air cleaner which is easily replaced and maximizes inflow and exhaust of air by a fan for air cleaning. In the related art, the filter assembly for an air cleaner includes a support which is formed in a wide plate shape and includes a plurality of through-holes through which air passes at a predetermined region about a center thereof, a drive unit which includes a motor mounted on one surface of the support and providing a rotating force for sucking air in a direction of the through-hole and a fan for generating an air flow by the rotating force of the motor, a filter unit which is in close contact with the other surface of the support and removes impurities of the air passing through the through-holes, and a support wall which is provided on the other surface of the support with which the filter is in close contact to extend to a predetermined height in a vertical direction with respect to the other surface to surround the predetermined region in which the plurality of through-holes are formed to support and fix the filter, in which the filter unit is inserted into the support wall of the support in an interference-fitting manner and is supported by the support wall.

In the related art, an air pressure is improved by the fan, and thus, even when the same rotating force is used, performance in air cleanness is improved, and a filter is very easily replaced.

However, in the related art, all polluted air passes through only the filter, and thus, in a case where the filter has two or more layers in order to improve a function of the filter, the air only moves through all the layers. Accordingly, there is a disadvantage that the air flow is improved only by using a stronger fan for rapid air purification.

The above information disclosed in this Background section is only for enhancement of understanding of the background of the described technology and therefore it may contain information that does not form prior art that is already known in this country to a person of ordinary skill in the art.

### SUMMARY

Accordingly, the present disclosure is directed to a compound filter module for a vehicle air cleaner that substantially obviates one or more of problems due to limitations and disadvantages of the prior art.

Additional features and advantages of the disclosure will be set forth in the description which follows and in part will be apparent from the description, or may be learned by practice of the disclosure. Other advantages of the present disclosure will be realized and attained by the structure particularly pointed out in the written description and claims hereof as well as the appended drawings.

In addition, the present disclosure provides a compound filter module for a vehicle air cleaner capable of increasing polluted air throughout to improve air purification efficiency while sucking, absorbing, and decomposing polluted air existing in a vehicle so as to discharge purified air.

In an aspect of the present disclosure, there is provided a compound filter module for a vehicle air cleaner which is inserted into a vehicle air cleaner to purify polluted air, including: an upper body in which at least one side of an upper portion is opened; an UV irradiation unit which is coupled to a lower portion of the upper body, includes a plurality of UV LEDs, and is configured to perform irradiation of UV from a lower body side; a disk-shaped photocatalyst cartridge which is located below the UV irradiation unit and includes a plurality of photocatalytic beads including a photocatalyst reacting with UV irradiated by the UV irradiation unit; a disk-shaped zeolite cartridge which is located below the photocatalyst cartridge and includes a plurality of zeolite beads adsorbs harmful substances from the polluted air sucked through the lower body; and the lower body which is located below the zeolite cartridge and in which at least one side of a lower portion is formed to be opened, in which each of the photocatalyst cartridge and the zeolite cartridge includes at least one through hole which is formed in an up-down direction.

In an aspect of the present disclosure, the photocatalyst cartridge may be formed in a hollow disk shape, include two or more radial frames which are provided radially from a central axis and an annular frame which is formed in a circumferential direction, and have two or more first space portions which are formed symmetrically by the radial frame and the annular frame and a second space portion which is formed to penetrate up and down in a slit shape inside the two or more radial frames, the zeolite cartridge may be formed in a hollow disk shape, includes two or more radial frames which are provided radially from a central axis and an annular frame which is formed in a circumferential direction, and have two or more third space portions which are formed symmetrically by the radial frame and the annular frame and a fourth space portion which is formed to penetrate up and down in a slit shape inside the two or more radial frames, and the plurality of photocatalytic beads may be densely disposed in the first space portion, and the plurality of zeolite beads may be densely disposed in the third space portion.

In an aspect of the present disclosure, a sum of surface areas of the second space portions may be the same as a sum of surface areas of the fourth space portions, and the sum of the surface areas of the second space portions may be 3 to 30 percent of a sum of surface areas of the first space portions and the sum of the surface areas of the second space portions.

In an aspect of the present disclosure, the third space portion may be formed to have the same size as the first space portion, the fourth space portion may be formed to have the same size as the second space portion, and the second space portion and the fourth space portion may be disposed at the same position in the up-down direction.

In an aspect of the present disclosure, the photocatalyst cartridge and the zeolite cartridge may respectively include a fifth space portion and a sixth space portion which are formed to penetrate up and down about the center axis.

In an aspect of the present disclosure, at least one of the photocatalyst cartridge and the zeolite cartridge may be rotatable about the center axis.

In an aspect of the present disclosure, the photocatalytic bead may be formed by coating a silicon oxide bead with titanium dioxide nanopowder adhered by an inorganic binder.

In an aspect of the present disclosure, the zeolite bead may be formed to have 2 times or more volume than that of the photocatalytic bead.

In an aspect of the present disclosure, a disk-shaped porous mesh disk may be provided between the UV irradiation unit and the photocatalyst cartridge, between the photocatalyst cartridge and the zeolite cartridge, and between the zeolite cartridge and the lower body, respectively.

It is to be understood that both the foregoing general description and the following detailed description are exemplary and explanatory and are intended to provide further explanation of the disclosure as claimed.

### BRIEF DESCRIPTION OF THE DRAWINGS

The accompanying drawings, which are included to provide a further understanding of the disclosure and are incorporated in and constitute a part of this application, illustrate aspects of the disclosure and together with the description serve to explain the principle of the disclosure.

In the drawings:

FIG. 1 is a perspective view of a compound filter module for a vehicle air cleaner according to an aspect of the present disclosure;

FIG. 2 is a perspective view when viewed in a different direction illustrating an air flow of the compound filter module for a vehicle air cleaner according to the aspect of the present disclosure;

FIG. 3 is an exploded perspective view of the compound filter module for a vehicle air cleaner according to the aspect of the present disclosure;

FIG. 4 is a schematic view illustrating an UV irradiation by an UV irradiation unit according to an aspect of the present disclosure;

FIG. 5 is a plan view of a photocatalyst cartridge and a zeolite cartridge according to an aspect of the present disclosure;

FIG. 6 is a cross-sectional view of the compound filter module for a vehicle air cleaner according to an aspect of the present disclosure;

FIG. 7 is a plan view of a photocatalyst cartridge and a zeolite cartridge according to another aspect of the present disclosure;

FIG. 8 is a perspective view of a photocatalyst cartridge and a zeolite cartridge according to another aspect of the present disclosure;

FIGS. 9 and 10 are cross-sectional views of a compound filter module for a vehicle air cleaner according to another aspect of the present disclosure; and

FIG. 11 is a graph illustrating an acetic acid removal result calculated by adjusting a bead density of the compound filter module for a vehicle air cleaner according to an aspect of the present disclosure.

### DETAILED DESCRIPTION

Hereinafter, aspects of the present disclosure will be described in detail with reference to the accompanying drawings. However, in descriptions of the present disclosure, when a technical idea of the present disclosure is obscured or unclear by specifically explaining the known configuration, the description of the known configuration will be omitted.

FIG. 1 is a perspective view of a compound filter module for a vehicle air cleaner according to an aspect of the present disclosure, FIG. 2 is a perspective view when viewed in a different direction illustrating an air flow of the compound filter module for a vehicle air cleaner according to the aspect of the present disclosure, FIG. 3 is an exploded perspective view of the compound filter module for a vehicle air cleaner according to the aspect of the present disclosure, and FIG. 4 is a schematic view illustrating an UV irradiation by an UV irradiation unit according to an aspect of the present disclosure.

A compound filter module 100 for a vehicle air cleaner according to the aspect of the present disclosure is a filter module which is inserted into a vehicle air cleaner to purify polluted air, in which the compound filter module 100 includes an upper body 110 in which at least one side of an upper portion is formed to be opened, a lower body 180 in which at least one side of a lower portion is formed to be opened, and two or more cartridges which are inserted into the upper body 110 and the lower body 180 and allow the polluted air inside the vehicle to be purified by each method.

In an example, the compound filter module 100 for a vehicle air cleaner according to the present aspect includes an UV irradiation unit 120 which is coupled to a lower portion of the upper body 110, includes a plurality of UV LEDs 122, and is configured to perform irradiation of UV from the lower body 180 side (direction), a disk-shaped photocatalyst cartridge 140 which is located below the UV irradiation unit 120 and includes a plurality of photocatalytic beads (not illustrated) including a photocatalyst reacting with UV emitted from the UV irradiation unit 120, and a disk-shaped zeolite cartridge 160 which is located below the photocatalyst cartridge 140 and includes a plurality of zeolite beads (not illustrated) adsorbs harmful substances from the polluted air sucked through the lower body 180. In this case, the lower body 180 is located below the zeolite cartridge 160.

In an example, each of the photocatalyst cartridge 140 and the zeolite cartridge 160 includes at least one through hole which is formed in an up-down direction.

When air sucked through the lower body 180 is purified while moving upward, the through hole increases the total amount of the moving air to promote a circulation of the air, and thus, rapid air purification can be realized. That is, when the polluted air passes through the photocatalyst cartridge 140 and zeolite cartridge 160, a passage speed is very low due to friction and collision between the polluted air, and the photocatalytic beads and the zeolite beads, and thus, the entire circulation of the air is slow. However, a portion of the air moves rapidly through the through hole, and thus, a circulation speed of the entire air can increase, and a throughput of polluted air increases.

The through hole will be described in detail later.

In an example, the photocatalytic bead is formed by coating a silicon oxide bead with titanium dioxide nanopowder adhered by an inorganic binder.

When the titanium dioxide (TiO2) is irradiated with UV (ultraviolet rays), an oxidation reaction is performed to oxidize and decompose organic compounds contained in the polluted air while realizing antibacterial and deodorization.

Incidentally, when the titanium dioxide is irradiated with UV, a photocatalytic reaction is realized. Reactive substances formed through the photocatalytic reaction destroy various bacteria or organic pollutants such as cigarette smoke, viruses, bacteria, and VOCs, and are discharged as water vapor and carbon dioxide. In an example, the reactive substance may be a hydroxy radical having a very large oxidizing power formed by reaction of water molecules with holes which are separated from electrons generated on a surface of an UV irradiation surface of a photocatalyst.

The titanium dioxide is an example of a photocatalytic material, and has an advantage that the titanium dioxide can be used semi-permanently because the titanium dioxide is not deformed during the UV irradiation compared to other photocatalytic materials such as ZnO, CdS, ZrO2, and V203.

In an example, the zeolite bead is made of a known zeolite, and is mainly made of a water-containing aluminum silicate mineral containing alkali metal or alkaline earth metal. The zeolite has a molecular sieve function and can adsorb a large amount of water at the same time through a large gap generated by breaking the rules of a network structure, and has property of selectively adsorbing an unsaturated hydrocarbon or a polar substance by the action of cations in a crystal structure. Accordingly, the zeolite can be used as carbon dioxide adsorption, removal of harmful substances, antibacterial properties, food quality maintenance, and freshness maintenance.

The zeolite bead according to the present aspect is formed by processing the zeolite into a bead shape, and when the polluted air collides and comes into contact with the plurality of zeolite beads disposed in the zeolite cartridge, the zeolite beads can adsorb harmful substances and carbon dioxide from the polluted air.

The compound filter module 100 for a vehicle air cleaner according to the aspect of the present disclosure may include disk-shaped porous mesh disks 130, 150, and 170 between the UV irradiation unit 120 and the photocatalyst cartridge 140, between the photocatalyst cartridge 140 and the zeolite cartridge 160, and between the zeolite cartridge 160 and the lower body 180, respectively.

Each of the porous mesh disks 130, 150, and 170 prevents the photocatalytic beads of the photocatalyst cartridge 140 and the zeolite beads of the zeolite cartridge 160 from escaping to the outside and allows the polluted air sucked through the lower body 180 to be rapidly discharged from the zeolite cartridge 160 through the photocatalyst cartridge 180 through the upper body 110. For this purpose, a mesh size of each of the porous mesh disks 130, 150, and 170 may be smaller than a size of the photocatalytic bead or the zeolite bead, and in the porous mesh disks 130, 150, and 170, the porous meshes having different mesh sizes according to the sizes of the photocatalytic bead and the zeolite bead may be provided at each location.

FIG. 5 is a plan view of the photocatalyst cartridge and the zeolite cartridge according to an aspect of the present disclosure, FIG. 6 is a cross-sectional view when the compound filter module for a vehicle air cleaner according to the aspect of the present disclosure is taken along line A-A′ of FIG. 5, and FIG. 11 is a graph illustrating an acetic acid removal result calculated by adjusting a bead density of the compound filter module for a vehicle air cleaner according to the aspect of the present disclosure.

As illustrated in FIG. 5, the photocatalyst cartridge 140 according to an aspect of the present disclosure is formed in a hollow disk shape and includes two or more radial frames 141 which are provided radially from a central axis and an annular frame 143 which is formed in a circumferential direction.

The photocatalyst cartridge 140 includes two or more first space portions 142 which are formed symmetrically by the radial frame 141 and the annular frame 143 and a second space portion 144 which is formed to penetrate up and down in a slit shape inside the two or more radial frames 141.

In FIG. 5, according to the present aspect, three radial frames 141, three first space portions 142, and three second space portions 144 are formed. However, the present disclosure is not limited to this, and the number of each of the radial frames 141, the first space portions 142, and the second space portions 144 may be two, or four or more.

The zeolite cartridge 160 according to the aspect of the present disclosure is formed in a hollow disk shape and includes two or more radial frames 161 which are provided radially from a central axis and an annular frame 163 which is formed in a circumferential direction (refer to FIGS. 7 and 8 described later).

The zeolite cartridge 160 includes two or more third space portions 162 which are formed symmetrically by the radial frame 161 and the annular frame 163 and a fourth space portion 144 which is formed to penetrate up and down in a slit shape inside the two or more radial frames 161.

In the present aspect, three radial frames 161, three third space portions 162, and three fourth space portions 164 are formed. However, the present disclosure is not limited to this, and the number of each of the radial frames 161, the third space portions 162, and the fourth space portions 164 may be two, or four or more.

In this case, the plurality of photocatalytic beads are densely disposed in the first space portion 142 and the plurality of zeolite beads are densely disposed in the third space portion 162.

A maximum integration density of each of the photocatalytic beads and the zeolite beads can be close to 68% by volume, and this dense disposition rapidly reduces a speed when the polluted air sucked through the lower body 180 by the fan passes through the zeolite cartridge 160 and the photocatalyst cartridge 140.

As described above, in the photocatalyst cartridge 140 and the zeolite cartridge 160 of the present aspect, a portion of the air rapidly moves upward through the through hole, and thus, the air flow can be smoothly performed while receiving the maximum amount of the UV emitted from the UV irradiation unit 120.

In an example, the third space portion 162 is formed to have the same size as the first space portion 142, the fourth space portion 164 is formed to have the same size as the second space portion 144, and the second space portion 144 and the fourth space portion 164 are disposed at the same position in the up-down direction.

In this case, the second space portion 144 and the fourth space portion 164 are disposed at the same position in the up-down direction, and thus, a slit-shaped through hole is formed to penetrate the photocatalyst cartridge 140 and the zeolite cartridge 16 in the up-down direction. Accordingly, air rapidly moves outward through the through hole.

An air flow in a center portion of the compound filter module 100 can be secured by the through hole, and thus, the air moving through the through hole having a slit shape pulls the air moving upward through the third space portion 162 and the first space portion 142. Accordingly, the air can be smoothly flowed.

The present inventor compared the compound filer module 100 of a vehicle air cleaner of the present aspect with a comparative example in which the through hole is not formed, and measured a flow rate of a circulating air and a decomposition rate.

TABLE 1

| | Measured | Required time | Decomposition | Decomposition |
|---|---|---|---|---|
| | flow rate | for one | rate for one | rate after four |
| | (m³/hour) | revolution | revolution (%) | hours elapse (%) |
| Filter module of | 5 | 12 | 4 | 43 |
| comparative example | | | | |
| Compound filter | 15 | 4 | 1.5 | 51 |
| module of the | | | | |
| present disclosure | | | | |

TABLE 2

| | Decomposition rate of filter module | Decomposition rate of compound filter |
|---|---|---|
| Hour | of comparative example (%) | module of the present aspect(%) |
| 30 minutes | 10 | 11 |
| One hour | 25 | 29 |
| Four hours | 43 | 51 |
| Eight hours | 65 | 80 |

The present experiment was conducted in accordance with a SPS-KACA002-132:2018 test method of Korea Institute for Construction and Living Environment Testing (KCL). A small-sized air cleaner having the compound filter module 100 and according to the present aspect and a small-sized air cleaner having a filter module of the comparative example were contained in a 1 m3 standard chamber, and were tested for filter efficiency according to ability to remove acetic acid (CH3COOH). (Fan 3500 rpm and a filter diameter 60 mm)

As a result of measuring a time until air cleaning inside the chamber was completed, the circulation of the entire air by the compound filter module 100 according to the present aspect was achieved within a short time of about 3 times, compared to the comparative example. The photocatalytic decomposition effect of the entire air in one revolution was slightly lower in the case of the compound filter module 100 than in the case of the comparative example, but when measured based on the same time, the photocatalytic decomposition effect was improved by about 18% in the case of the compound filter module 100.

That is, it can be seen that when the vehicle air cleaner including the compound filter module 100 according to the present aspect is continuously utilized, a better photocatalytic effect can be obtained than in the prior art.

In an example, a sum of surface areas of the second space portions 144 is configured to be equal to a sum of surface areas of the fourth space portions 164, and the sum of the surface areas of the second space portions 144 is configured to be 3 to 30 percent of the sum of the surface areas of the first space portions 142 and the sum of the surface areas of the second space portions 144.

In a case where the sum of the surface areas of the second space portions 144 is configured to be 3 to 30 percent of the sum of the surface areas of the first space portions 142 and the sum of the surface areas of the second space portions 144, that is, 3 to 30 percent of the total surface areas, it is possible to increase the circulation effect of the entire air by the air moving upward from below through the through hole. In a case where the sum of the surfaces of the second space portions 144 is maintained at 5 to 15 percent of the total surface areas, it is possible to obtain an optimal circulation effect of the entire air.

In an example, the zeolite bead may be formed to have 2 times or more volume than that of the photocatalytic bead. According to this structure, the speed of the air flow is partially reduced while air permeability of the polluted air sucked from the lower portion of the zeolite cartridge 160 is maintained, and thus, a residence time of the air flowing into the photocatalyst cartridge 140 can increase.

Each of the zeolite bead or the photocatalytic bead may be formed in a spherical bead having a single size. In a case where the spherical beads having the single size are most densely disposed in the zeolite cartridge 160 and the photocatalyst cartridge, theoretically, the spherical beads can be densely disposed up to 73 percent, but, actually, can be densely disposed at a level of 68 to 70 percent.

Accordingly, in a case where the density of the beads is reduced by 5 to 15 percent like the above-described aspect in which the sum of the surface areas of the second space portions 144 is maintained at 5 to 15 percent of the total surface area, it is possible to obtain the optimal circulation effect of the entire air.

That is, in a case where the density of the beads is 59.5 to 64.6 percent, it is possible to obtain the above-described optimal circulation effect of the entire air.

When in the density of the beads is reduced, it is difficult to uniformly and densely dispose the beads, and there is a problem that a noise may occur due to movements of the beads. However, in a case where the density of the beads is reduced by a certain degree, it is possible to obtain the optimal circulation effects of the entire air as illustrated in FIG. 11.

FIG. 7 is a plan view of a photocatalyst cartridge and a zeolite cartridge according to another aspect of the present disclosure, FIG. 8 is a perspective view of the photocatalyst cartridge and the zeolite cartridge according to another aspect of the present disclosure, FIG. 9 is a cross-sectional view when a compound filter module for a vehicle air cleaner according to another aspect of the present disclosure is taken along line B-B′ of FIG. 7, and FIG. 10 is a cross-sectional view when the compound filter module for a vehicle air cleaner according to another aspect of the present disclosure is taken along a center line.

In the compound filter module 100 for a vehicle air cleaner according to another aspect of the present disclosure, at least one of the photocatalyst cartridge 140 and the zeolite cartridge 160 is configured to be rotatable about a center axis.

The rotation of the photocatalyst cartridge 140 and/or the zeolite cartridge 160 adjusts a circulation speed of the entire air, and increases or decreases a pollution reduction function according to a situation such as a pollution degree of the air.

For example, as illustrated in FIGS. 7 to 9, when the zeolite cartridge 160 is rotated by 30°, a portion of air purified by the zeolite beads while passing through the third space 162 of the zeolite cartridge 160 can be rapidly moved upward through the second space portion 144 of the photocatalyst cartridge 140. In another example, the air sucked through the fourth space portion 164 of the zeolite cartridge 160 may be purified by the photocatalytic beads while moving through the first space portion 142 of the photocatalyst cartridge 140.

Compared to a case where the air flowing into the third space portion 162 passes through the first space portion 142 and the fourth space portion 164 and the second space portion 144 are formed to pass through with each other, in the partial pollution purification with respect to partial air, the pollution reduction effect during a primary circulation of the air is relatively low. However, by increasing the circulation speed of the entire air, it is possible to increase the pollution reduction effect during the same time.

A protrusion (not illustrated) may be formed in the zeolite cartridge 160 and/or the photocatalyst cartridge 140 so that the cartridges can rotate about the center axis.

A user can perform adjustment so that the zeolite cartridge 160 and/or the photocatalyst cartridge 140 are rotated in consideration of a pollution degree of the air inside the vehicle and a use time of the vehicle and a suitable purification of the polluted air can be realized.

In another example, the compound filter module 100 for a vehicle air cleaner may further includes a separate drive unit (not illustrated) and a separate control unit (not illustrated) so as to perform a control so that the cartridge is automatically rotated based on the measured pollution degree of the air.

In the compound filter module for a vehicle air cleaner according to still another aspect of the present disclosure, the photocatalyst cartridge 140 and the zeolite cartridge 160 respectively include a fifth space portion 146 and a sixth space portion 166 which are formed to penetrate up and down about the center axis.

The fifth space portion 146 and the sixth space portion 166 replaces or complement the through hole when a size of the through hole formed by the second space portion 144 and the fourth space 164 is too small or the through hole almost disappears due to the rotation of any one of the photocatalyst cartridge 140 and the zeolite cartridge 160.

That is, even when any one of the photocatalyst cartridge 140 and the zeolite cartridge 160 rotates about the center axis, the fifth space portion 146 and the sixth space portion 166 always forms the through hole in the up-down direction, and thus, it is possible to increase the circulation speed of the entire air.

Hereinbefore, the present disclosure is described in detail with reference to specific aspects. However, since the aspects are merely examples for making the present disclosure easy to understand, substitutions, additions, and modifications are made within a scope which does not depart from a technical spirit of the present disclosure and are included in a protection scope of the present disclosure as defined by the following claims.

According to the present disclosure, the polluted air throughout increases, and thus, the air inside the vehicle is effectively purified.

In addition, according to the present disclosure, the polluted air throughout is adjusted, and thus, the air is appropriately purified according to a situation.

It will be apparent to those skilled in the art that various modifications and variations can be made in the compound filter module for a vehicle air cleaner of the present disclosure without departing from the spirit or scope of the aspects. Thus, it is intended that the present disclosure covers the modifications and variations of the aspects provided they come within the scope of the appended claims and their equivalents.

What is claimed is:

1. A compound filter module for a vehicle air cleaner which is inserted into a vehicle air cleaner to purify polluted air, comprising:
an upper body in which at least one side of an upper portion is opened;
an UV irradiation unit which is coupled to a lower portion of the upper body, includes a plurality of UV LEDs, and is configured to perform irradiation of UV from a lower body side;
a disk-shaped photocatalyst cartridge which is located below the UV irradiation unit and includes a plurality of photocatalytic beads including a photocatalyst reacting with UV emitted from the UV irradiation unit;
a disk-shaped zeolite cartridge which is located below the photocatalyst cartridge and includes a plurality of zeolite beads adsorbs harmful substances from the polluted air sucked through the lower body,
wherein the lower body which is located below the zeolite cartridge and in which at least one side of a lower portion is formed to be opened,
wherein each of the photocatalyst cartridge and the zeolite cartridge includes at least one through hole which is formed in an up-down direction.

2. The compound filter module of claim 1, wherein the photocatalyst cartridge is formed in a hollow disk shape, includes two or more radial frames which are provided radially from a central axis and an annular frame which is formed in a circumferential direction, and has two or more first space portions which are formed symmetrically by the radial frame and the annular frame and a second space portion which is formed to penetrate up and down in a slit shape inside the two or more radial frames,
wherein the zeolite cartridge is formed in a hollow disk shape, includes two or more radial frames which are provided radially from a central axis and an annular frame which is formed in a circumferential direction, and has two or more third space portions which are formed symmetrically by the radial frame and the annular frame and a fourth space portion which is formed to penetrate up and down in a slit shape inside the two or more radial frames, and
wherein the plurality of photocatalytic beads are densely disposed in the first space portion, and the plurality of zeolite beads are densely disposed in the third space portion.

3. The compound filter module of claim 2, wherein a sum of surface areas of the second space portions is equal to a sum of surface areas of the fourth space portions, and
wherein the sum of the surface areas of the second space portions is 3 to 30 percent of a sum of surface areas of the first space portions and the sum of the surface areas of the second space portions.

4. The compound filter module of claim 2, wherein the third space portion is formed to have the same size as the first space portion, the fourth space portion is formed to have the same size as the second space portion, and the second space portion and the fourth space portion are disposed at the same position in the up-down direction.

5. The compound filter module of claim 2, wherein the photocatalyst cartridge and the zeolite cartridge respectively include a fifth space portion and a sixth space portion which are formed to penetrate up and down about the center axis.

6. The compound filter module of claim 2, wherein at least one of the photocatalyst cartridge and the zeolite cartridge is rotatable about the center axis.

7. The compound filter module of claim 1, wherein the photocatalytic bead is formed by coating a silicon oxide bead with titanium dioxide nanopowder adhered by an inorganic binder.

8. The compound filter module of claim 1, wherein the zeolite bead is formed to have 2 times or more volume than that of the photocatalytic bead.

9. The compound filter module of claim 1, further comprising a disk-shaped porous mesh disk respectively provided between the UV irradiation unit and the photocatalyst cartridge, between the photocatalyst cartridge and the zeolite cartridge, and between the zeolite cartridge and the lower body.

10. A compound filter module for purifying polluted air in a vehicle, comprising:
an upper body having at least one side of an upper portion being opened;
an UV irradiation unit coupled to a lower portion of the upper body and including a plurality of UV LEDs,
a lower body emitting UV irradiated from the UV irradiation unit;
a disk-shaped photocatalyst cartridge located below the UV irradiation unit and including a plurality of photocatalytic beads having a photocatalyst reacting with the UV emitted from the UV irradiation unit;
a disk-shaped zeolite cartridge located below the disk-shaped photocatalyst cartridge and including a plurality of zeolite beads adsorbing harmful substances from the polluted air sucked through the lower body,
wherein the disk-shaped photocatalyst cartridge has a hollow disk shape, includes two or more radial frames which are provided radially from a central axis and an annular frame which is formed in a circumferential direction, and has two or more first space portions which are formed symmetrically by the radial frame and the annular frame and a second space portion which is formed to penetrate up and down in a slit shape inside the two or more radial frames, and
wherein the disk-shaped zeolite cartridge has a hollow disk shape, includes two or more radial frames which are provided radially from a central axis and an annular frame which is formed in a circumferential direction, and has two or more third space portions which are formed symmetrically by the radial frame and the annular frame and a fourth space portion which is formed to penetrate up and down in a slit shape inside the two or more radial frames.

11. The compound filter module of claim 10, wherein the plurality of photocatalytic beads are densely disposed in the first space portion and the plurality of zeolite beads are densely disposed in the third space portion.

12. The compound filter module of claim 10, wherein a sum of surface areas of the second space portions is equal to a sum of surface areas of the fourth space portions.

13. The compound filter module of claim 12, wherein the sum of the surface areas of the second space portions is 3 to 30 percent of a sum of surface areas of the first space portions and the sum of the surface areas of the second space portions.

14. The compound filter module of claim 10, wherein the two or more third space portion is formed to have a same size as the first space portion, the fourth space portion is formed to have a same size as the second space portion, and the second space portion and the fourth space portion are disposed at the same position in the up-down direction.

15. The compound filter module of claim 10, wherein the disk-shaped photocatalyst cartridge and the disk-shaped zeolite cartridge respectively include a fifth space portion and a sixth space portion which are formed to penetrate up and down about the center axis.

16. The compound filter module of claim 10, wherein at least one of the disk-shaped photocatalyst cartridge and the disk-shaped zeolite cartridge is rotatable with respect to the center axis.

17. The compound filter module of claim 10, wherein the plurality of photocatalytic beads are formed by coating a silicon oxide bead with titanium dioxide nanopowder adhered by an inorganic binder.

18. The compound filter module of claim 10, wherein the plurality of zeolite beads have at least two times greater in volume that of the plurality of photocatalytic beads.

19. The compound filter module of claim 10, further comprising a disk-shaped porous mesh disk respectively provided between the UV irradiation unit and the photocatalyst cartridge, between the photocatalyst cartridge and the zeolite cartridge, and between the zeolite cartridge and the lower body.
